(19) United States
(12) Patent Application Publication
(10) Pub. No.: US 2011/0166192 A1
(43) Pub. Date: Jul. 7, 2011

(54) PROCESS FOR CHARGING A POLYMERIC PRODUCT WITH ATTRIBUTE IMPARTING AGENT(S)

(75) Inventors: Preeti LODHA, Mumbai (IN); Bir KAPOOR, Mumbai (IN); Tushar MAHAJAN, Maharashtra (IN); Anasuya SAHOO, Dhenkanal (IN)

(73) Assignee: ADITYA BIRLA SCIENCE & TECHNOLOGY CO. LTD., Maharashtra (IN)

(21) Appl. No.: 13/045,106

(22) Filed: Mar. 10, 2011

Related U.S. Application Data
(63) Continuation of application No. PCT/IN09/00501, filed on Sep. 11, 2009.

(30) Foreign Application Priority Data
Sep. 12, 2008 (IN) 1946/MUM/2008

(51) Int. Cl. A01N 43/80; D01F 1/10; D03D 15/00; D03D 1/00; D04H 13/00; A01N 31/14; A01N 53/08; A01P 1/00; A01P 3/00; A01P 7/04; A01P 7/02; C08L 33/00; C08K 5/01; A61K 8/97; A61Q 13/00; C04B 16/02; C04B 16/04

(52) U.S. Cl. 514/372; 264/211; 442/187; 442/333; 514/721; 514/531; 524/556; 512/5; 106/501.1

(57) ABSTRACT

The invention relates to a process for charging a polymeric product with attribute imparting agent(s) to the polymeric product prepared by the process and to a composition for preparing the polymeric product capable of retaining the acquired attributes for a long period of time.

### CROSS-REFERENCE TO RELATED APPLICATION

This application is a continuation of International Application no. PCT/IN2009/000501, filed Sep. 11, 2009, which claims the benefit of India Patent Application No. 1946/MUM/2008, filed Sep. 12, 2008, the disclosures of which are hereby incorporated by reference.

### FIELD OF INVENTION

The invention relates to a process for charging a polymeric product with an attribute imparting agent, to the polymeric product prepared by the process and to a composition for preparing the polymeric product.

### BACKGROUND

Polymeric products having attributes are desired for various applications. To impart such attributes, the polymeric products are usually treated with active ingredients possessing such attributes. Conventionally, prior to such treatment, the polymer product is applied with a surface finish, to enable binding of the active ingredients. However, retention of such active ingredients over the finish is usually poor and the polymeric products lose their activity upon subsequent treatments such as washing, bleaching and the like.

US 2008/003218 disclose a method of applying active ingredients, to polymer dopes, in microencapsulated form. By this method of application, though the active ingredients are retained in the fiber for a longer period of time, the ingredients, once discharged out of the encapsulant, cannot be replenished with the active ingredients, thus reducing the effectiveness thereof.

It is desirable to have a process for charging polymeric products with attribute imparting agents that would result in charged polymeric products that would retain the acquired attributes for a long period of time.

### SUMMARY OF THE INVENTION

In accordance with the present invention there is provided a process for charging a polymeric product with attribute imparting agent(s), the process comprising first contacting a polymeric dope or its precursors thereof with an emulsion containing an unencapsulated non-polar organic reservoir to form a spinnable composition comprising the dope and the emulsion, then spinning the composition through a die to form a regenerated composite carrying the reservoir capable of prolonged retention of attribute imparting agents(s) and finally contacting the said composite or product thereof with a formulation comprising at least one attribute-imparting agent and a swelling agent at a temperature equal to or greater than the melting point of the reservoir for at least 15 minutes and at a pressure in the range of 1 to 5 bars.

Typically, the polymeric dope is selected from a group consisting of viscose dope, acrylic dope, and cellulose dope.

Typically, the organic reservoir is a paraffin hydrocarbon, organic alcohol, organic fatty acid or an ester thereof, polyethylene or any mixture thereof having melting point in the range of 10-120° C.

Preferably, the composite is contacted with an aqueous formulation comprising 0.0001 to 15% of attribute imparting agent(s), 0.01 to 99.999% of swelling agent(s) and 0.0001 to 15% of a surfactant selected from the group consisting of ionic surfactant, non-ionic surfactant, amphoteric surfactant and a polymeric surfactant.

Typically, the attribute imparting agent is at least one agent selected from the group consisting of an antimicrobial agent, a fungicide, a bactericide, an algicide, therapeutic agents, a aromatic compound, an insecticide, an acaricide, nematicide, an anti-wrinkle agent, an emollient, an antioxidant, a flame retardant, a filler, a reinforcing agent, a UV protective agent, a plasticizer, a water transporting/absorbing agent, oil soluble vitamins, organic dyes and colourants.

Typically, the swelling agent(s) is at least one selected from the group consisting of water, cyclohexanol, n-amyl amine, n-amyl alcohol, 1,5-diaminopentane, glycerol, Phenol, Benzyl alcohol, m-toluidine, m-cresol, 2-ethyl phenol, Benzyl amine, Aniline, Ethyl aniline, Polystyrene, Esters/phenols, Acetonitrile, o-Nitrophenol, p-Nitrophenol, Dimethyl formamide, dimethyl sulfoxide, o-phenylphenol, Ethylene carbonate, propylene carbonate, nitromethane, iso-amyl alcohol, potassium hydroxide, Cellosolve, Methyl Cellosolve, iso propyl alcohol, Propyl alcohol and m-cresol.

Preferably, the ratio of the weight of the composite to the weight of formulation is in the range of 1:1 to 1:500, preferably in the range of 1:5 to 1:100.

In accordance with the present invention a charged polymeric product prepared by a process comprising first contacting a polymeric dope or its precursors thereof with an emulsion containing an unencapsulated non-polar organic reservoir adapted to retain one or more attribute imparting agent(s) having >1% solubility in a non-polar organic solvent to form a spinnable composition comprising the dope and the emulsion, then spinning the composition through a die to form a regenerated composite carrying the reservoir capable of prolonged retention of attribute imparting agents(s) and finally contacting said composite or product thereof with a formulation comprising at least one attribute-imparting agent and a swelling agent at a temperature equal to or greater than the melting point of the reservoir for at least 15 minutes and at a pressure in the range of 1 to 5 bars.

Preferably, the attribute imparting agent is retained by up to 60% of the total mass of the reservoir and the retained attribute imparting agent.

Typically, the attribute imparting agent(s) is at least one selected from the group consisting of an antimicrobial agent, a fungicide, a bactericide, an algicide, therapeutic agents, a perfumery compound, an insecticide, an acaricide, nematicide, an anti-wrinkle agent, an emollient, an antioxidant, a flame retardant, a filler, a reinforcing agent, a UV protective agent, a plasticizer, a water transporting/absorbing agent, oil soluble vitamins, organic dyes and colourants.

The polymeric product provided in accordance with the present invention is capable of sustained release/controlled release of attribute imparting agent(s).

Preferably, the release of the attribute imparting agent(s) is controlled by temperature.

Preferably, a composition for preparing a polymeric product capable of prolonged retention of acquired attribute(s), the composition comprising a polymer dope and an unencapsulated non-polar organic reservoir having melting point in the range of 10° C. to 120° C.

Preferably, the polymer dope is at least one selected from a viscose dope containing 7-15% of cellulose in xanthate form, a cellulose dope containing 5-25% cellulose in N-methyl morphopline oxide, or an ionic liquid having the formula I

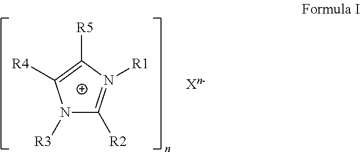

wherein Ri and R₃ are each, independently of one another, an organic rest having 1 to 20 carbon atoms, R₂, R₄ and R₅ are each, independently of one another, an H atom or an organic radical having from 1 to 20 carbon atoms, X is an anion such as halide, a diethyl phosphate, or a carboxylate anion of formula R₃—COO— and Rₐ is a C₁ to C₂₀ alkyl group and n is 1, 2 or 3 and an acrylic dope selected from an acrylic polymer, a modacrylic polymer or a blend thereof.

An article prepared from the polymeric product as disclosed herein includes a woven or non-woven or a product made therefrom.

### BRIEF DESCRIPTION OF THE ACCOMPANYING DRAWINGS

The invention will now be described with reference to accompanying drawings, in which

FIG. 1 depicts a graph wherein the representation shows the sustained/control release of perfumery substance in the polymeric product at various temperatures; and

FIG. 2 depicts a graph wherein the representation shows the sustained/control release of an anti-bacterial agent in the polymeric product at various temperatures.

### DETAILED DESCRIPTION OF THE INVENTION

Accordingly, the invention provides a process for charging a polymeric product capable of prolonged retention of acquired attribute(s).

When used throughout the specification the following terms have the meaning indicated:

The term “attribute imparting agent” as used herein refers to atom(s), ion(s), groups of atoms or of ions, a compound or mixture of compounds capable of imparting attribute such as colour, smell and the like or of imparting functional attributes such as antimicrobial activity, UV radiation screening, wrinkle resistance and the like.

The term acquired attribute as used herein refers to the attribute acquired from the attribute imparting agent.

The term “non polar organic reservoir” as used herein refers to a high molecular weight hydrocarbon or derivatives thereof including paraffin wax, stearyl alcohol, Myristyl alcohol and the like.

The term “encapsulated” as used herein refers to enclosed in a polymeric shell.

The term “unencapsulated” as used herein refers to not completely enclosed in a polymeric shell.

The term “dope” as defined herein would mean a solution, slurry or a polymer composition.

In one embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying the reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s)and finally contacting the said composite or product thereof with a formulation comprising at least one attribute-imparting agent and a swelling agent at a temperature greater than or equal to the melting/softening point of the reservoir for at least 10 minutes wherein the polymeric dope is selected from a group consisting of viscose dope, acrylic dope, and cellulose dope.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes wherein the non-polar organic reservoir is a paraffin hydrocarbon, organic alcohol, organic fatty acid or an ester thereof, polyethylene or any mixture thereof having melting point in the range of 10-120° C.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes wherein the composite is contacted with an aqueous formulation comprising 0.0001 to 15% of attribute-imparting agent(s), 0.01 to 99.999% of swelling agent and 0.0001 to 15% of a surfactant.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes wherein the surfactant is selected from a group consisting of ionic surfactant, non-ionic surfactant, amphoteric surfactant and polymeric surfactant.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes wherein the attribute imparting agent is an antimicrobial agent, a fungicide, a bactericide, an algicide, therapeutic agents, a aromatic compound, an insecticide, an acaricide, nematicide, an anti-wrinkle agent, an emollient, an antioxidant, a flame retardant, a filler, a reinforcing agent, a UV protective agent, a plasticizer, a water transporting/absorbing agent, oil soluble vitamins, organic dyes, colourants or any mixture thereof.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes wherein the swelling agent is selected from a group consisting of water, cyclohexanol, n-amyl amine, n-amyl alcohol, 1,5-diaminopentane, glycerol, Phenol, Benzyl alcohol, m-toluidine, m-cresol, 2-ethyl phenol, Benzyl amine, Aniline, Ethyl aniline, Polystyrene, Esters/phenols, Acetonitrile, o-Nitrophenol, p-Nitrophenol, Dimethyl formamide, dimethyl sulfoxide, o-phenylphenol, Ethylene carbonate, propylene carbonate, nitromethane, iso-amyl alcohol, potassium hydroxide, Cellosolve, Methyl Cellosolve, iso propyl alcohol, Propyl alcohol and m-cresol.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a regenerated composite carrying non-polar organic reservoir capable of prolonged retention of attribute imparting agents(s) and finally contacting the said composite or product thereof with a formulation comprising at least one attribute-imparting agent and a swelling agent at a temperature greater than to equal to the melting/softening point of the reservoir for at least 10 minutes wherein ratio of the weight of the composite to the weight of formulation is in the range of 1:1 to 1:500.

In another embodiment, the invention provides a process for charging a polymeric product with attribute imparting agent(s), the process comprising first contacting a polymeric dope or its precursor thereof with an emulsion containing an unencapsulated non-polar organic reservoir to form a spinnable composition comprising the dope and the emulsion, then spinning the composition through a die/spinneret to form a composite carrying non-polar organic reservoir capable of prolonged retention of attribute imparting agents(s) and finally contacting the said composite or product thereof with a formulation comprising at least one attribute-imparting agent and a swelling agent at a temperature greater than or equal to the melting/softening point of the reservoir for at least 10 minutes wherein the ratio is in the range of 1:5 to 1:100.

In another embodiment, the invention provides a charged polymeric product prepared by a process comprising first contacting a polymeric dope or its precursors thereof with an emulsion containing an unencapsulated non-polar organic reservoir adapted to retain one or more attribute imparting agent(s) having >1% solubility in a non-polar organic solvent to form a spinnable composition comprising the dope and the emulsion, then spinning the composition through a die to form a regenerated composite carrying the reservoir capable of prolonged retention of attribute imparting agents(s) and finally contacting said composite or product thereof with a formulation comprising at least one attribute-imparting agent and a swelling agent at a temperature equal to or greater than the melting point of the reservoir for at least 15 minutes and at a pressure in the range of 1 to 5 bars.

In another embodiment, the invention provides a polymeric product capable of prolonged retention of acquired attribute(s), the product comprising unencapsulated non-polar organic reservoir retaining at least one attribute imparting agent wherein the attribute imparting agent is retained by up to 60% of the total mass of the reservoir and the retained attribute imparting agent.

In another embodiment, the invention provides a polymeric product capable of prolonged retention of acquired attribute(s), the product comprising unencapsulated non-polar organic reservoir retaining at least one attribute imparting agent wherein the attribute imparting agent is an antimicrobial agent, a fungicide, a bactericide, an algicide, therapeutic agents, a perfumery compound, an insecticide, an acaricide, nematicide, an anti-wrinkle agent, an emollient, an antioxidant, a flame retardant, a filler, a reinforcing agent, a UV protective agent, a plasticizer, a water transporting/absorbing agent, oil soluble vitamins, organic dyes, colourants or any mixture thereof.

In another embodiment, the invention provides a polymeric product retaining the acquired attribute(s) after laundering for >20 washings following AATCC procedure in the presence of AATCC standard reference detergent in a laundering machine.

In another embodiment, the invention provides a polymeric product adapted to retain the acquired attribute(s) after bleaching, dyeing, sulfurising, curing or resin treatment.

In another embodiment, the invention provides a polymeric product capable of prolonged retention of acquired attribute(s) wherein the polymeric product is capable of sustained/controlled release of attribute imparting agent(s).

In another embodiment, the invention provides a polymeric product capable of prolonged retention of acquired attribute(s) wherein the release of attribute imparting agent(s) is controlled by temperature.

In another embodiment, the invention provides a composition for preparing a polymeric product capable of prolonged retention of acquired attribute(s), the composition comprising a polymer dope and an unencapsulated non-polar organic reservoir having melting point in the range of 10° C. to 120° C.

In another embodiment, the invention provides a composition for preparing a polymeric product capable of prolonged retention of acquired attribute(s), the composition comprising a polymer dope and an unencapsulated non-polar organic reservoir having melting point in the range of 10° C. to 120° C. wherein the polymer dope is a viscose dope containing 7-15% of cellulose in xanthate form.

In another embodiment, the invention provides a composition for preparing a polymeric product capable of prolonged retention of acquired attribute(s), the composition comprising a polymer dope and an unencapsulated non-polar organic reservoir having melting point in the range of 10° C. to 120° C. wherein the polymer dope is a cellulose dope containing 5-25% cellulose N-methyl morphopline oxide or an ionic liquid having the formula I

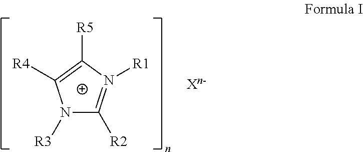

wherein R₁ and R₃ are each, independently of one another, an organic rest having 1 to 20 carbon atoms, R₂, R₄ and R₅ are each, independently of one another, an H atom or an organic radical having from 1 to 20 carbon atoms, X is an anion such as halide, a carboxylate anion of formula Rₐ—COO— and Rₐ is a C₁ to C₂₀ alkyl group and n is 1, 2 or 3.

In another embodiment, the invention provides a composition for preparing a polymeric product capable of prolonged retention of acquired attribute(s), the composition comprising a polymer dope and an unencapsulated non-polar organic reservoir having melting point in the range of 10° C. to 120° C. wherein the polymer dope is an acrylic dope selected from an acrylic polymer, a mono acrylic polymer or a blend thereof.

In another embodiment, the invention provides a woven, knit or non-woven fabric prepared from the polymeric product.

In another embodiment, the invention provides a woven, knit or non-woven fabric prepared from >5% of the polymeric product blended with other natural, manmade or synthetic fibres such as polyester, cotton, wool, lycra, silk and linen.

The invention provides a polymeric product having unencapsulated non-polar organic that helps in retaining the acquired attribute(s) of the polymeric product for a long period of time. The attribute(s) are acquired by treatment of the polymeric product with an attribute imparting agent. Typical attribute imparting agents include, but are not limited to, antimicrobial agents including parabens such as methyl paraben, ethyl paraben, propyl paraben and butyl paraben, thaizole compounds such as 4,5-dichloro-n-octyl-4-isothiazoline-3-one (DCOIT), 2-n-octyl-4-isothiazolin-3-one (OIT), Benzisothiazolinone (BIT), imidazole based compounds such as 1-[(2-chlorophenyl)(diphenyl)methyl]-1H-imidazole (cloitrimazole), Halogen-O-hydroxy-diphenyl compounds such as 5-chloro-2-(2,4-dichlorophenoxy)phenol (triclosan), 2,4,4′-trichloro-2′-acetoxy-diphenyl ether (triclosan acetate), Thiocarbamates such as o-(2-naphthyl) methyl (3-methylphenyl)thiocarbamate (tolnaftate), Diiodo methyl p-tolyl sulfone, Phenol derivatives such as pentachlorophenol, chloroquinaldol, 4-chloro-3,5-xylenol, Phenoxyethanol, 1-phenoxy propan-2-ol, Chlorhexidine and derivatives thereof, Trihalocarbanilide derivatives, compounds commonly used in perfumery including phenolic compounds; essential oils; aldehydes; ketones; polycyclic compounds; esters; and alcohols, Woody perfume ingredients including cedarwood oil (essential oil), guaicwood oil (essential oil), gamma ionone (ketone), sandalwood oil (essential oil), and methyl cedryl ketone (ketone), Sweet perfume ingredients including coumarin (ketone), vanillin (4 hydroxy-3methoxy benzaldehyde) (aldehyde), ethyl maltol (Alcohol), phenyl acetaldehyde (aldehyde), heliotropin (aldehyde), acetophenone (ketone), and dihydrocoumarin (ketone), Citrus perfume ingredients including orange oil (essential oil), lemon oil (essential oil), citral (aldehyde), beta methyl naphthyl ketone (ketone), terpinyl acetate (ester), nonyl aldehyde (aldehyde), terpineol (alcohol), and dihydromyrcenol (alcohol), Floral perfume ingredients including a variety of floral subcategories, such as rose, lavender, jasmin, and muguet, Rose perfume ingredients including geranyl acetate (ester), geraniol (alcohol), citronellyl acetate (ester), phenyl ethyl alcohol (alcohol), alpha damascone (ketone), beta damascone (ketone), geranium oil (essential oil), and natural rose oil (essential oil), Lavender perfume ingredients including dihydro terpinyl acetate (ester), ethyl hexyl ketone (ketone), lavandin (essential oil), lavender (essential oil), tetra hydro linalool (alcohol), linalool (alcohol), and linalyl acetate (ester), Jasmin perfume ingredients including benzyl acetate (ester), butyl cinnamic aldehyde (aldehyde), methyl benzoate (ester), natural jasmin oil (essential oil), methyl dihydro jasmonate (ester), Muguet perfume ingredients including cycalmen aldehyde (aldehyde), benzyl salycilate (ester), hydroxycitronellol (alcohol), citronellyl oxyacetaldehyde (aldehyde), and hydroxy aldehyde (aldehyde), Fruity perfume ingredients including ethyl-2-methyl butyrate (ester), allyl cyclohexane propionate (ester), amyl acetate (ester), ethyl acetate (ester), gamma decalactone (ketone), octaiactone (ketone), undecalactone (aldehyde), ethyl aceto acetate (ester), benzaldehyde (aldehyde), Animal perfume ingredients including methyl phenyl acetate (ester), indol (2,3, benzpyrrole) (phenolic), creosol (phenolic), iso butyl quinolin (phenolic), and androstenol (phenolic), Spice perfume ingredients including anisic aldehyde (aldehyde), aniseed (essential oil), clove oil (essential oil), eugenol (phenolic), iso eugenol (phenolic), thymol (phenolic), anethol (phenolic), cinnamic alcohol (alcohol), and cinnamic aldehyde (aldehyde), Green perfume ingredients including beta gamma hexenol (alcohol), brom styrol (alcohol), dimethyl benzyl carbinol (alcohol), methyl heptine carbonate (ester), cis-3-hexenyl acetate (ester), and galbanum oil (essential oil), Musk perfume ingredients including galaxolide (phenol), cyclopentadecanolide (phenol), musk ketone (ketone), ambrettolide (phenol), tonalid (phenol), and ethylene brassylate (ester), Balsamic perfume ingredients including fir balsam (essential oil), peru balsam (essential oil), and benzoin resinoid (essential oil), Mint perfume ingredients including laevo carvone (ketone), menthol (alcohol), methyl salicylate (ester), peppermint oil (essential oil), spearmint oil (essential oil), eucalyptus (essential oil), anisyl acetate (ester), methyl chavicol (alcohol), Chemical perfume ingredients including benzyl alcohol (alcohol), diproplene glycol (alcohol), ethanol (alcohol), and benzyl benzoate (ester), Andrane, Cedramber, Decyl methyl ether, Galaxolide, Grisalva, Indolarome-solid, Orange flower ether, Ozofleur, Phenafleur, Tobacarol, Paracresyl methyl Ether, Karanal, Cyclogalbanate, Piconia, Iso-cyclemone, Iso E super, Celestolide-solid, Fleuramone, dihydro isojasmone, Isojasmone, Tonalid, Methyl Ionone, Dulcinyl-solid, Acetanisole, Vetikone, Undecylenic Aldehyde, lilial, Vanillin, Cinnamic Alcohol, Iso-Eugenol, Tetra-Hydro Geraniol, Calone, Dihydro-isojasmone, Galaxolide, Karanal, Yara Yara (Nerolin), Decyl methyl ether, Ethyl Methyl Phenyl Glycidate, Para-Cresyl Phenyl acetate, Undecalactone, Clonal IFF, insecticides/insect repellant such as Pyrethroids, Organothiophospahte based insecticides such as malathion, pirimiphos methyl, Organochlorine compounds such as dichlorodiphenyltrichloroethane, Neonicotinoids, icaridin, essential oils such as Citronella Java Oil, Lemon grass oil, Cedar wood oil, Tea Tree Oil, Eucalyptus Oil, Acaricides such as permethrin, Oil soluble Vitamins such as Vitamin A (retinol, palmitate), Vitamin D precursor (ergosterol), Vitamin E (Tocopherols-alpha-tocopherol and tocoperol acetate, & tocotrienols), Vitamin E acetate and Vitamin K (Menaquinone, 2-methyl-1,4-naphthoquinone derivatives and Phylloquinone), Emollients or skin moisturizers such as Aloe Vera, Skin lightening agents such as 4-butyl-1,3-dihydroxy benzene (slightly soluble in water), Anti-wrinkle or anti-ageing molecules such as 1,4-Bis (3,4-dihydroxyphenyl)-2-3-dimethylbutane, UV resistant Chemicals such as 2-hydroxy-4-methoxy benzophenone, 2-Ethylhexyl-2-cyano-3,3-diphenylacrylate, octyl triazone, octyl methoxy cinnamate, 4-t-butyl-4′-methoxydibenzoylmethane. The attribute imparting agent can also be a mold release agent, an antioxidant, a flame retardant, filler, a reinforcing agent, a UV protective agent, a plasticizer, a water transporting/absorbing agent or a cationic dyeability agent.

The invention also provides articles and products prepared from a polymeric product having prolonged acquired attributes. Polymeric product described herein can be used in the production of yarns, woven fabrics, carpets and nonwoven fabrics. Fabrics made from the polymeric product described herein can be used in a variety of end-use applications, like in the manufacture of consumer products, hygiene products, medical products, operating room products including gowns, drapes, sterile wraps, footcovers, towels and related articles. Other exemplary products that can be produced from fabrics made from the polymeric product described herein include active wears, hosiery/socks, rubber gaskets and seals, fiberfills, feminine care products, diapers and incontinence products.

In the examples and the results that will follow the various tests were conducted as per the procedure given below.

The activity of an anti-bacterial agent in the polymeric product was conducted by Qualitative AATCC 147 method. The test was conducted with Staphylococcus Aureus bacteria strain ATCC 6538. Observation of growth under the specimen accompanied with no zone of inhibition was considered as No (i.e. failed test with no anti-bacterial activity) and no growth under the specimen, with or without zone of inhibition was considered as Yes (passed test with anti-bacterial activity).

The activity of perfume in the various samples of the polymeric product was analyzed by an expert panel trained by a Professional Perfumer to distinguish the presence of odor note by comparing it with the odor note perceived in the pure perfume. If > or =70% panelists could identify the perfume note in the sample, it was considered as Yes (good efficacy) and <70% could identify the perfume note in the sample, it was considered as No (failed efficacy).

The activity of mosquito repellency in the polymeric product was measured by reduction in number of landings measured as per the given protocol. A piece of the polymeric product is placed on top of a mosquito cage of 30×30×30 cm covered with gauze. A bowl filled with water of 38±1° C. is put on the piece of tissue. The warmed surface area measured 65 cm² and plays the role of an enticement. The mosquito cage contained 100 female mosquitoes of the species Aedes aegypti. The number of mosquitoes sitting on the warmed tissue was counted after 1 and 5 minutes. Four independent countings are done. The same procedure was performed using various test-samples. A separate population of mosquitoes is used for each test-sample. The reduction of landings after 5 minutes is given as the final result. The reduction in landings of > and =30% was considered as Yes (has the desired efficacy) and <30% was considered as No (does not have desired efficacy).

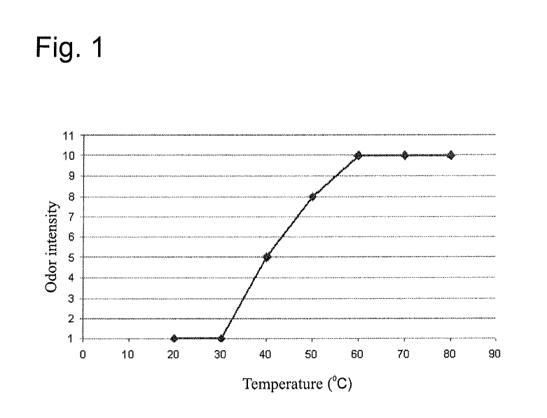

For the release of attribute imparting agent was incorporated with tonalid perfumery ingredient. The product was washed and dried at ambient temperature of 20° C., and was maintained at said temperature for 30 minutes and measured for efficacy. The study of sustained/control release of perfumery substance in the polymeric product of the invention was carried out at various temperatures. The results are displaced in FIG. 1 of accompanying drawings. From the FIG. 1, odor intensity analysis on a scale of 1-10 by a perfumery panel, where 1 is minimum odor and 10 is strongest odor. It is clear that the perfumery substance in the polymeric product of the invention has a sustained/control release profile with temperature.

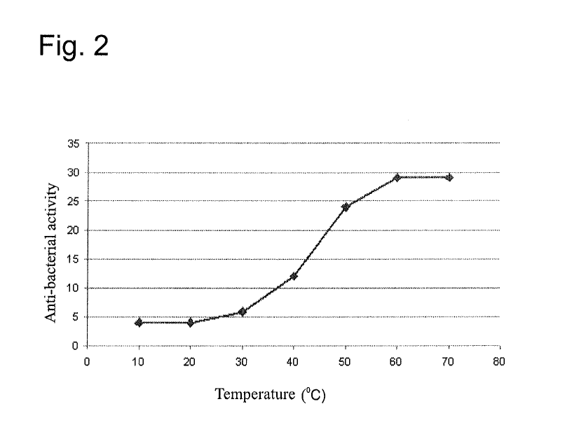

Also the polymeric product containing reservoir (melting range of 35-65° C.) was incorporated with triclosan as anti-bacterial ingredient. The product was washed and dried at ambient temperature of 20° C. after which the fabric was maintained at said temperature for 30 minutes and measured for efficacy. Anti-bacterial analysis was done based on AATCC 147 with 24 hr contact time and the zone of inhibition (in mm) was measured. The sustained/control release of an anti-bacterial agent in the polymeric product was studied at various temperatures. The results are displaced in FIG. 2 of accompanying drawings. From the FIG. 2, it is clear that the anti-bacterial agent in the polymeric product of the invention has a sustained/control release profile with temperature.

The invention is further illustrated by way of the following non-limiting examples.

### EXAMPLE 1

### Preparation of Polymeric Products Having Prolonged Retention of Acquired Attributes

(a) A Composition for Preparing Polymeric Product:

Paraffin wax emulsion was prepared by adding the 10 g a non-polar organic reservoir (Of the type indicated in column 2 of table 2, for example, paraffin wax) to a 4 g non-ionic surfactant with HLB 15, Hydol 20 and 20 g water at 70□C to obtain a stable emulsion using ultrasonicator. The emulsion was added to a polymer dope (As indicated in column 3 of table 3, for example viscose dope containing 9% cellulose in xanthate form) and 6% alkali.

(b) Preparation of a Polymeric Product Capable of Prolonged Retention of Acquired Attributes:

The composition as prepared in Example 1(a) was spun through a spinneret into a sulfuric acid and aluminum sulfate/zinc sulfate spin bath and processed as per the standard spinning procedures to obtain composite comprising paraffin reservoir. The polymeric products prepared are displayed in Table 2 (Sr. Nos 1 to 20).

(c) Treatment of Polymeric Product:

100 g of fabric prepared from the polymeric product obtained as in Example 1(b) is contacted with 1 liter of solution containing the attribute imparting agent at a ratio by weight of 1:10 at 70° C. for 120 minutes. Subsequently the fabric is washed at 70° C. with 2 gpl standard detergent for 30 minutes followed by hot and cold water wash for 15 minutes each.

### EXAMPLE 2

### Conventional Method for Preparation of Polymeric Product Comprising Attribute Imparting Agent

The preparation of polymeric product and the treatment of the polymeric product was carried out by the procedure as in examples 1 (b) and 1(c) except that the polymeric dope is not contacted with any non-polar organic reservoir, before spinning, to form the product. The polymeric products conventionally prepared are displayed in Table 2 (at Sr. Nos. 11 to 18) and the treated conventional polymeric products are displayed in Table 1 (at Sr. Nos. 22 to 29).

### EXAMPLE 3

### Test for Retention of Attribute(s) Acquired by the Polymeric Product

To test the retention of attributes imparted by the attribute imparting agent, the polymeric product samples prepared according to example 1 and example 2 were subjected to laundering for upto 20 washings in the presence of an AATCC standard detergent at a temperature in the range of 30 to 70° C. in a laundering machine, following the AATCC procedure. Test for retention was carried out also by treatments such as bleaching; disperse dyeing reactive dyeing, sanforizing, resin treatment and curing of polymeric product samples as indicated in column 5 of table 1. The retention of attributes in the polymeric product after such treatments were determined and displayed in Table 1.

TABLE 1
Treatment of composite prepared according to Example 1(b) with attribute imparting agent(s)
and study of their retention as compared to retention in conventional polymeric products

| | | | | | | Formulation (F) containing attribute imparting agent | | | |
|---|---|---|---|---|---|---|---|---|---|
| | Non | | | | | | | | Retention of |
| | polymer | Polymer | Process Description | | | Attribute | | Laundering | Attribute |
| Sr. | organic | dope/Product | | | P:F | imparting | Surfactant | or other | after |
| No | reservoir | blend | T° C. | t min | ratio | agent (%) | (%) | treatment | treatment |
| 1 | Paraffin | 100% | 70 | 120 | 10:1 | Triclosan | Ionic | 20 wash at | Yes |
| | wax | Viscose | | | | (0.1%) | (0.1%) | 30° C. | |
| 2 | Paraffin | 100% | 70 | 120 | 10:1 | Triclosan | Ionic | Bleaching | Yes |
| | wax | Viscose | | | | (0.1%) | (0.1%) | | |
| 3 | Paraffin | 100% | 70 | 120 | 10:1 | Triclosan | Ionic | Disperse | Yes |
| | wax | Viscose | | | | (0.1%) | (0.1%) | Dyeing | |
| 4 | Paraffin | 100% | 70 | 120 | 10:1 | Triclosan | Ionic | Reactive | Yes |
| | wax | Viscose | | | | (0.1%) | (0.1%) | Dyeing | |
| 5 | Paraffin | 100% | 70 | 120 | 10:1 | Triclosan | Ionic | Sanforizing | Yes |
| | wax | Viscose | | | | (0.1%) | (0.1%) | | |
| 6 | Paraffin | 100% | 70 | 120 | 10:1 | Triclosan | Ionic | Resin | Yes |
| | wax | Viscose | | | | (0.1%) | (0.1%) | Treatment | |
| | | | | | | | | & Curing | |
| 7 | Paraffin | 100% | 70 | 120 | 500:1 | Triclosan | Ionic | 1 wash at | Yes |
| | wax | Viscose | | | | (0.0001%) | (0.1%) | 70° C. | |
| 8 | Paraffin | 100% | 70 | 120 | 10:1 | Triclosan | Ionic | 1 wash at | Yes |
| | wax | Viscose | | | | (10.0%) | (0.1%) | 70° C. | |
| 9 | Paraffin | 100% | 70 | 120 | 10:1 | Woody | Ionic | 1 wash at | Yes |
| | wax | Viscose | | | | perfume | (0.1%) | 70° C. | |
| | | | | | | (0.1%) | | | |
| 10 | Paraffin | 100% | 70 | 120 | 10:1 | Diiodemethyl | Ionic | 1 wash at | No |
| | wax | Viscose | | | | tolylsulphone | (0.1%) | 70° C. | |
| | | | | | | (insoluble | | | |
| | | | | | | active) | | | |
| | | | | | | (0.1%) | | | |
| 11 | Paraffin | 100% | 70 | 120 | 10:1 | Octyl | Ionic | 1 wash at | No |
| | wax | Viscose | | | | isothiazolinone | (0.1%) | 70° C. | |
| | | | | | | (insoluble | | | |
| | | | | | | active) | | | |
| | | | | | | (0.1%) | | | |
| 12 | Paraffin | 100% | 70 | 120 | 10:1 | Triclosan + | Ionic + | 1 wash at | Yes |
| | wax | Viscose | | | | Permetrin + | Non- | 70° C. | |
| | | | | | | Woody Perfume | ionic | | |
| | | | | | | (0.3%) | (0.3%) | | |
| 13 | Paraffin | 10% Viscose + | 70 | 120 | 10:1 | Triclosan | Ionic | 1 wash at | Yes |
| | wax | 90% | | | | (0.1%) | (0.1%) | 70° C. | |
| | | polyester | | | | | | | |
| 14 | Paraffin | 10% Viscose + | 70 | 120 | 10:1 | Triclosan | Ionic | 1 wash at | Yes |
| | wax | 90% cotton | | | | (0.1%) | (0.1%) | 70° C. | |
| 15 | Paraffin | 100% | 70 | 120 | 25:1 | Permethrin | Ionic | 1 wash at | Yes |
| | Wax | Acrylic | | | | (0.1%) | (0.1%) | 70° C. | |
| 16 | Paraffin | 100% | 70 | 120 | 25:1 | Transfluthrin | Ionic | 1 wash at | Yes |
| | Wax | Acrylic | | | | (0.1%) | (0.1%) | 70° C. | |
| 17 | Paraffin | 100% | 70 | 120 | 10:1 | Permethrin | Ionic | 1 wash at | Yes |
| | Wax | Cellulose | | | | (0.1%) | (0.1%) | 70° C. | |
| | | (made using | | | | | | | |
| | | ionic liquid) | | | | | | | |
| 18 | Paraffin | 100% | 70 | 120 | 25:1 | Transfluthrin | Ionic | 1 wash at | Yes |
| | Wax | Cellulose | | | | (0.1%) | (0.1%) | 70° C. | |
| | | (made using | | | | | | | |
| | | ionic liquid) | | | | | | | |
| 19 | Paraffin | 100% | 70 | 120 | 10:1 | Triclosan + | Ionic + | 1 wash at | Yes |
| | wax + | Viscose | | | | Permetrin + | Nonionic | 70° C. | |
| | stearyl | | | | | Woody Perfume | (0.3%) | | |
| | alcohol = | | | | | (0.3%) | | | |
| | 5:1 | | | | | | | | |
| 20 | Stearyl | 100% | 60 | 120 | 10:1 | Octyl | Ionic | 1 wash at | Yes |
| | alcohol | Viscose | | | | isothiazolinone | (0.1%) | 70° C. | |
| | | | | | | (0.1%) | | | |
| 21 | Myristyl | 100% | 70 | 120 | 10:1 | Octyl | Ionic | 1 wash at | Yes |
| | alcohol | Viscose | | | | isothiazolinone | (0.1%) | 70° C. | |
| | | | | | | (0.1%) | | | |
| 22 | — | 100% | 70 | 120 | 10:1 | Triclosan | Ionic | 20 wash at | No |
| | | Viscose | | | | (0.1%) | (0.1%) | 30° C. | |
| 23 | — | 100% | 70 | 120 | 10:1 | Woody | Non- | 1 wash at | No |
| | | Viscose | | | | Perfume | ionic | 70° C. | |
| | | | | | | (0.1%) | (0.1%) | | |
| 24 | — | 100% Cotton | 70 | 120 | 10:1 | Triclosan | Ionic | 1 wash at | No |
| | | | | | | (0.1%) | (0.1%) | 70° C. | |
| 25 | — | 100% | 70 | 120 | 10:1 | Permethrin | Ionic | 1 wash at | No |
| | | Acrylic | | | | (1%) | (1%) | 70° C. | |
| 26 | — | Viscose with | 70 | 120 | 10:1 | Triclosan | Ionic | 1 wash at | No |
| | | encapsulated | | | | (1%) | (1%) | 70° C. | |
| | | reservoir | | | | | | | |
| 27 | | Viscose with | 70 | 120 | 10:1 | Floral | Ionic | 1 wash at | No |
| | | encapsulated | | | | Perfume | (1%) | 70° C. | |
| | | reservoir | | | | (1%) | | | |
| 28 | | Acrylic with | 70 | 120 | 10:1 | Triclosan | Ionic | 1 wash at | No |
| | | encapsulated | | | | (1%) | (1%) | 70° C. | |
| | | reservoir | | | | | | | |
| 29 | | Acrylic with | 70 | 120 | 10:1 | Floral | Ionic | 1 wash at | No |
| | | encapsulated | | | | Perfume | (1%) | 70° C. | |
| | | reservoir | | | | (1%) | | | |

All examples with Viscose and Cellulose use water as swelling agent and with Acrylic use benzyl alcohol as swelling agent.

Ionic liquid used for above cellulose examples is ethyl methyl imidazolium acetate.

TABLE 2
Various polymeric products prepared by the process in Example 1

| Sr. | | | | % of attribute |
|---|---|---|---|---|
| No | Paraffin type | Composite/Product | Attribute imparting agent | imparting agent^{†} |
| 1 | Paraffin wax | 100% | Triclosan | 0.1 |
| | | Viscose | | |
| 2 | Paraffin wax | 100% | Triclosan | 0.0001 |
| | | Viscose | | |
| 3 | Paraffin wax | 100% | Triclosan | 10.0 |
| | | Viscose | | |
| 4 | Paraffin wax | 100% | Woody Perfume | 0.1 |
| | | Viscose | | |
| 5 | Paraffin wax | 100% | Triclosan + Permetrin + Woody | 0.3 |
| | | Viscose | Perfume | |
| 6 | Paraffin wax | 10% Viscose + | Triclosan | 0.1 |
| | | 90% | | |
| | | polyester | | |
| 7 | Paraffin wax | 10% Viscose + | Triclosan | 0.1 |
| | | 90% cotton | | |
| 8 | Paraffin | 100% | Triclosan + Permetrin + Woody | 0.3 |
| | wax + stearyl | Viscose | Perfume | |
| | alcohol = 5:1 | | | |
| 9 | Stearyl | 100% | Octyl isothiazolinone | 0.1 |
| | alcohol | Viscose | | |
| 10 | Myristyl | 100% | Octyl isothiazolinone | 0.1 |
| | alcohol | Viscose | | |
| 11 | — | 100% | Triclosan | 0.1 |
| | | Viscose | | |
| 12 | — | 100% | Woody Perfume | 0.1 |
| | | Viscose | | |
| 13 | — | 100% Cotton | Triclosan | 0.1 |
| 14 | — | 100% | Permethrin | 1 |
| | | Acrylic | | |
| 15 | — | 100% | Triclosan | 1 |
| | | Viscose with | | |
| | | encapsulated | | |
| | | reservoir | | |
| 16 | — | 100% | Floral Perfume | 1 |
| | | Viscose with | | |
| | | encapsulated | | |
| | | reservoir | | |
| 17 | — | 100% | Triclosan | 1 |
| | | Acrylic with | | |
| | | encapsulated | | |
| | | reservoir | | |
| 18 | — | 100% | Floral Perfume | 1 |
| | | Acrylic with | | |
| | | encapsulated | | |
| | | reservoir | | |

^{†}The percentage displayed is the percentage of attribute imparting agent in the solution used for treating the composite.

From table, it is clear that for the polymeric products of the invention (Sample Nos 1 to 10) wherein polymeric products comprise non polar organic reservoir display retention of attributes after laundering or other treatments. Further the polymeric products of the invention are capable of being recharged with the attribute imparting agent as evident from table 2. Prolonged retention in the attribute/activity is advantageous from the perspective of quality and cost. The retention in attribute/activity results from the retention in the attribute imparting agent in the paraffin reservoir. Prolonged retention of the attribute imparting agent provides materials that are durable and suitable for preparing quality articles and products. The prolonged retention of the attribute imparting agent also improves the shelf life of the articles and products prepared from the polymeric products.

1. A process for charging a polymeric product with attribute imparting agent(s), the process comprising first contacting a polymeric dope or its precursors thereof with an emulsion containing an unencapsulated non-polar organic reservoir to form a spinnable composition comprising the dope and the emulsion, then spinning the composition through a die to form a regenerated composite carrying the reservoir capable of prolonged retention of attribute imparting agents(s) and finally contacting the said composite or product thereof with a formulation comprising at least one attribute-imparting agent and a swelling agent at a temperature equal to or greater than the melting point of the reservoir for at least 15 minutes and at a pressure in the range of 1 to 5 bars.

2. The process as claimed in claim 1, wherein the polymeric dope is selected from a group consisting of viscose dope, acrylic dope, and cellulose dope.

3. The process as claimed in claim 1, wherein the organic reservoir is a paraffin hydrocarbon, organic alcohol, organic fatty acid or an ester thereof, polyethylene or any mixture thereof having melting point in the range of 10-120° C.

4. The process as claimed in claim 1, wherein the composite is contacted with an aqueous formulation comprising 0.0001 to 15% of attribute imparting agent(s), 0.01 to 99.999% of swelling agent(s) and 0.0001 to 15% of a surfactant selected from the group consisting of ionic surfactant, non-ionic surfactant, amphoteric surfactant and a polymeric surfactant.

5. The process as claimed in claim 1, wherein the attribute imparting agent is at least one agent selected from the group consisting of an antimicrobial agent, a fungicide, a bactericide, an algicide, therapeutic agents, a aromatic compound, an insecticide, an acaricide, nematicide, an anti-wrinkle agent, an emollient, an antioxidant, a flame retardant, a filler, a reinforcing agent, a UV protective agent, a plasticizer, a water transporting/absorbing agent, oil soluble vitamins, organic dyes and colourants.

6. The process as claimed in claim 1, wherein the swelling agent(s) is at least one selected from the group consisting of water, cyclohexanol, n-amyl amine, n-amyl alcohol, 1,5-diaminopentane, glycerol, Phenol, Benzyl alcohol, m-toluidine, m-cresol, 2-ethyl phenol, Benzyl amine, Aniline, Ethyl aniline, Polystyrene, Esters/phenols, Acetonitrile, o-Nitrophenol, p-Nitrophenol, Dimethyl formamide, dimethyl sulfoxide, o-phenylphenol, Ethylene carbonate, propylene carbonate, nitromethane, iso-amyl alcohol, potassium hydroxide, Cellosolve, Methyl Cellosolve, iso propyl alcohol, Propyl alcohol and m-cresol.

7. The process as claimed in claim 1, wherein the ratio of the weight of the composite to the weight of formulation is in the range of 1:1 to 1:500, preferably in the range of 1:5 to 1:100.

8. A charged polymeric product prepared by a process comprising first contacting a polymeric dope or its precursors thereof with an emulsion containing an unencapsulated non-polar organic reservoir adapted to retain one or more attribute imparting agent(s) having >1% solubility in a non-polar organic solvent to form a spinnable composition comprising the dope and the emulsion, then spinning the composition through a die to form a regenerated composite carrying the reservoir capable of prolonged retention of attribute imparting agents(s) and finally contacting said composite or product thereof with a formulation comprising at least one attribute-imparting agent and a swelling agent at a temperature equal to or greater than the melting point of the reservoir for at least 15 minutes and at a pressure in the range of 1 to 5 bars.

9. The polymeric product as claimed in claim 8, wherein the attribute imparting agent is retained by up to 60% of the total mass of the reservoir and the retained attribute imparting agent.

10. The polymeric product as claimed in claim 8, wherein the attribute imparting agent(s) is at least one selected from the group consisting of an antimicrobial agent, a fungicide, a bactericide, an algicide, therapeutic agents, a perfumery compound, an insecticide, an acaricide, nematicide, an anti-wrinkle agent, an emollient, an antioxidant, a flame retardant, a filler, a reinforcing agent, a UV protective agent, a plasticizer, a water transporting/absorbing agent, oil soluble vitamins, organic dyes and colourants.

11. The polymeric product as claimed in claim 8, capable of sustained release/controlled release of attribute imparting agent(s).

12. The polymeric product as claimed in claim 8, wherein the release of the attribute imparting agent(s) is controlled by temperature.

13. A composition for preparing a polymeric product capable of prolonged retention of acquired attribute(s), the composition comprising a polymer dope and an unencapsulated non-polar organic reservoir having melting point in the range of 10° C. to 120° C.

14. The composition as claimed in claim 13, wherein the polymer dope is at least one selected from a viscose dope containing 7-15% of cellulose in xanthate form, a cellulose dope containing 5-25% cellulose in N-methyl morphopline oxide, or an ionic liquid having the formula I 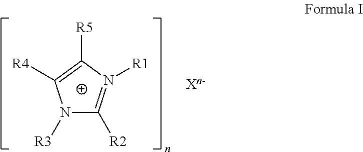
wherein Ri and R₃ are each, independently of one another, an organic rest having 1 to 20 carbon atoms, R₂, R₄ and R₅ are each, independently of one another, an H atom or an organic radical having from 1 to 20 carbon atoms, X is an anion such as halide, a diethyl phosphate, or a carboxylate anion of formula R₃—COO— and Rₐ is a C₁ to C₂₀ alkyl group and n is 1, 2 or 3 and an acrylic dope selected from an acrylic polymer, a modacrylic polymer or a blend thereof.

15. An article prepared from the polymeric product as claimed in the claim 8, wherein said article includes a woven or non-woven or a product made therefrom.
